(19) United States
(12) Patent Application Publication
(10) Pub. No.: US 2002/0042644 A1
(43) Pub. Date: Apr. 11, 2002

(54) Bifurcated fabric sleeve stent graft with junction region strengthening elements

(76) Inventor: E. Skott Greenhalgh, Wyndmoor, PA (US)

Correspondence Address: John A. Chionchio, Esquire, Synnestvedt & Lechner LLP, Suite 2600, 1101 Market Street, Philadelphia, PA, 19107-2950, US

(21) Appl. No.: 09/974,482

(22) Filed: Oct. 10, 2001

Related U.S. Application Data
(60) Provisional application No. 60/238,983, filed on Oct. 10, 2000.

(51) Int. Cl. A61F 2/06

(52) U.S. Cl. 623/1.13; 623/1.35; 623/1.51

(57) ABSTRACT

A bifurcated sleeve formed of interlaced filamentary members and having two or more flexible tubes joined together at a junction region is disclosed. The junction region is also formed of interlaced filamentary members and reinforced by the presence of elongated strengthening elements interlaced with the filamentary members forming the junction region. The strengthening elements may be multiple filamentary members which are plied or single filamentary members which have a relatively larger denier or increased tensile strength. The sleeve may be woven, knitted or braided. When knitted, the strengthening elements may be laid in or interknitted, and a locking stitch or a denser knit may be used at the junction region.

### RELATED APPLICATION

This application is based on and claims priority of U.S. Provisional Application No. 60/238,983, filed Oct. 10, 2000.

### FIELD OF THE INVENTION

This invention relates to stent grafts comprising bifurcated fabric sleeves reinforced at the junction region to prevent failure of the fabric at or near the point of bifurcation.

### BACKGROUND OF THE INVENTION

Bifurcated fabric sleeves may be woven, knitted or braided and comprise tubular structures, wherein a single tube branches into two or more branch tubes at a bifurcation point defined by a junction region located between the branch tubes where they connect to one another.

Both woven and knitted bifurcated sleeves find application in the construction of stent grafts for the repair of aortic aneurysms. An aneurysm is a pathologic dilation of a segment of a blood vessel which constitutes a weakened portion of the vessel. In a fusiform aneurysm 10, such as can occur in the abdominal aorta 12 as seen in FIG. 1, the entire circumference of the vessel is dilated and weakened. The majority of these aortic aneurysms are located in the distal abdominal aorta between the renal arteries 14 and the bifurcation point 16 where the abdominal aorta splits into the common iliac arteries 18.

Such aortic aneurysms constitute a serious condition, as an acute rupture of the aneurysm is fatal unless an emergency operation is performed. However, even when such operations are performed in time, the mortality rate is still greater than 50%.

Modern methods of treatment for aortic aneurysms focus on providing a stent graft which is positioned within the artery at the aneurysm. As seen in FIG. 1, stent graft 20 comprises a bifurcated fabric sleeve 22 forming the graft. Sleeve 22 may be woven, knitted or braided and has one end 24 which is attached to the inner surface of the artery above the aneurysm 10. The opposite end 26 of the bifurcated sleeve is split into two branch tubes 26a and 26b and has a junction region 28 comprising an extended area between the branch tubes which joins them together. The branch tubes 26a and 26b are attached to the inside surfaces of the iliac arteries 18 below the aneurysm 10. The stent graft 20 replaces the abdominal aorta in the region of the aneurysm 10, relieving the pressure on the weakened arterial wall and avoiding a potentially fatal rupture.

In relieving the pressure on the aneurysm, the bifurcated sleeve is subject to millions of hemodynamic pressure pulses over the lifetime of the patient as blood is pumped by the heart through the body. The pressure pulses put considerable stress on the sleeve at the junction region, trying to tear it apart. Furthermore, the junction region 28 is a natural stress concentration point as a result of the joining of the branch tubes at an acute angle. The stress concentration magnifies the stress in the junction region and may cause accelerated fatigue and subsequent failure of the graft there. Failure of the graft can have fatal consequences as pressure could be put back on the aneurysm, causing it to rupture, the patient bleeding to death unless treated in time.

It would clearly be advantageous to provide a bifurcated sleeve having greater resistance to failure at the junction region for use as a graft in the repair of aneurysms, as well as for other applications where a long fatigue life is required.

### SUMMARY AND OBJECTS OF THE INVENTION

The invention concerns a stent graft comprising a bifurcated sleeve formed of interlaced filamentary members. The sleeve comprises an elongated flexible first tubular member and at least one elongated flexible second tubular members joined to the first tubular member. A junction region, also formed of the interlaced filamentary members, is positioned between the first and second tubular members joining them together. The second tubular member may be joined to the first tubular member near its end or intermediately along its length. An elongated strengthening element having a relatively greater tensile strength than the filamentary members is interlaced with the filamentary members for reinforcing the junction region.

The bifurcated sleeve also has another elongated strengthening element having a relatively greater tensile strength than the filamentary members for reinforcing the junction region. This other strengthening element is preferably interlaced with the filamentary members and oriented angularly with respect to the aforementioned strengthening element, both of the strengthening elements intersecting one another within the junction region to provide reinforcement. Preferably, one of the strengthening elements is positioned substantially lengthwise along one of the first and second tubular members while the other traverses the junction region substantially perpendicularly to one of the first and second tubular members.

The filamentary members and the strengthening elements are preferably interlaced by weaving but may also be knitted or braided. There are various options available for providing strengthening elements having higher tensile strength. They may, for example, comprise plied filamentary members having substantially the same denier and made of substantially the same material as the filamentary members comprising the sleeve. They may also comprise a reinforcing filamentary member formed of a material having a relatively greater tensile strength than the material forming filamentary members comprising the sleeve. The strengthening elements may also comprise a reinforcing filamentary member having a relatively greater denier than the filamentary members comprising the sleeve.

It is an object of the invention to provide a bifurcated sleeve having a reinforced junction region for use in a stent graft.

It is another object of the invention to provide a bifurcated sleeve having an improved fatigue life.

It is again another object of the invention to provide a bifurcated sleeve having increased strength without increasing the bulk of the sleeve significantly.

These and other objects and advantages will become apparent upon consideration of the following drawings and detailed description of the preferred embodiments.

### BRIEF DESCRIPTION OF THE DRAWINGS

FIG. 1 shows a partial sectional view of an aortic aneurysm repaired by a stent graft;

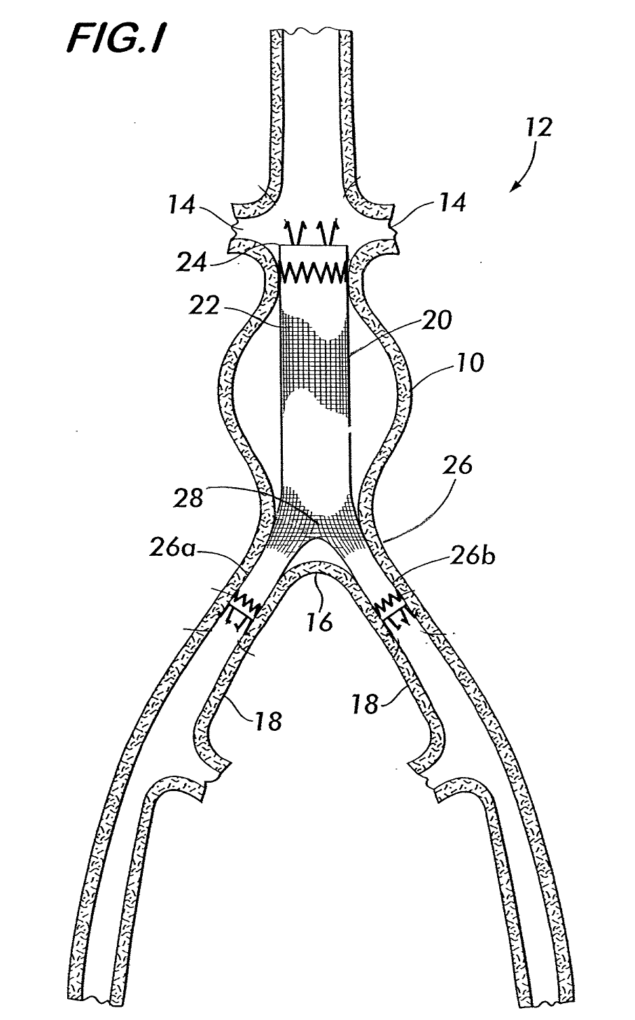

FIG. 2 shows a front view of a graft having strengthening elements according to the invention;

FIG. 3 shows a front view of a graft having an alternate embodiment of the strengthening elements according to the invention;

FIG. 4 shows a warp knit pattern on an enlarged scale illustrating the alternate embodiment of the strengthening element shown in FIG. 3; and

FIG. 5 shows a warp knit pattern on an enlarged scale illustrating another embodiment of the strengthening elements shown in FIG. 3.

### DETAILED DESCRIPTION OF THE PREFERRED EMBODIMENTS

Two bifurcated sleeve types are used extensively in the treatment of aneurysms. The woven bifurcated sleeve is preferred for use with endovascular stent grafts which are implanted in the artery through the use of a catheter. Woven grafts are preferred for this application because the endovascular stent graft must have as little bulk as possible and be readily collapsible to fit within the lumen of a catheter which, in turn, must fit within the lumen of the artery. Woven structures inherently have relatively minimal bulk when compared to knitted or braided structures having the same dimensions.

For vascular stent grafts which are implanted by more invasive surgical techniques, the bulk of the graft is not of primary concern, and knitted graft structures are preferred due to their inherent flexibility and compliance.

The bifurcated sleeve with junction region strengthening elements according to the invention is readily applicable to either woven or knitted bifurcated sleeves, as described below for both embodiments.

### Woven Bifurcated Sleeve Having Junction Region Strengthening Elements

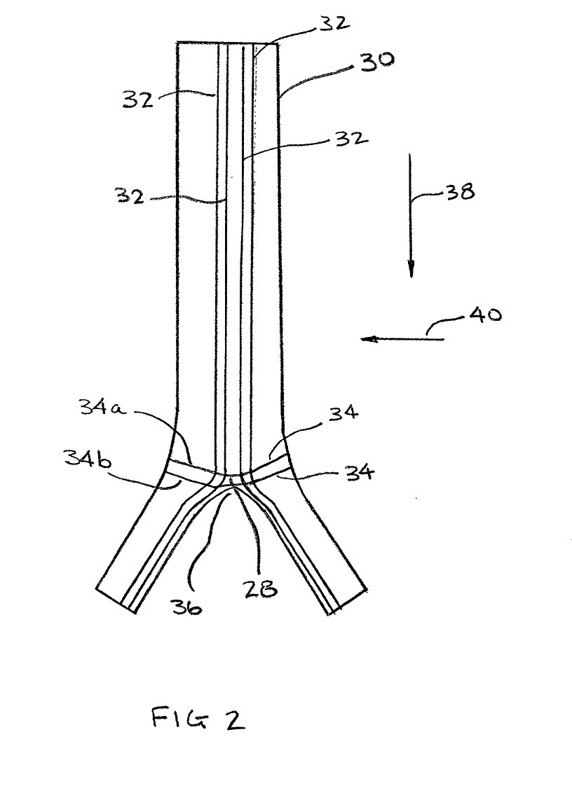

As shown in FIG. 2, the woven bifurcated sleeve 30 according to the invention has elongated strengthening elements 32 and 34 judiciously positioned so as to reinforce the known weak point, the junction region 28. The strengthening elements 32 and 34 preferably comprise elongated filamentary members which have a higher tensile strength than the other filamentary members and which are integrally woven into the sleeve during the weaving process.

It is advantageous to provide the strengthening elements in both the warp direction 38, as well as the fill direction 40 and have them intersect within the junction region. Since fabric sleeves are typically woven with the warp direction coinciding with the long axis of the sleeve as seen in FIG. 2, elements 32 can be considered warp strengthening elements and elements 34 fill strengthening elements.

The warp strengthening elements 32 run the length of the sleeve 30 and are arranged to intersect the junction region 28 by feeding them through the appropriate heddles on the loom which correspond to the region in the fabric where the junction region 28 will be formed during weaving. Preferably, both the warp strengthening elements 32 and fill strengthening elements 34 are interwoven on both the front face of the bifurcated sleeve (shown in FIG. 2) as well as the back face (not shown) to provide symmetric reinforcement and strengthening to the junction region 28.

The fill strengthening elements 34 are interwoven by manipulating the shuttle or the equivalent component on a shuttleless loom. How the shuttle is manipulated to effect the interweaving is determined largely by the type of strengthening element used, as described below.

The simplest and also the preferred strengthening elements 32 and 34 for both the warp and fill directions comprise plied yarns of the same denier and material as the rest of the yarns forming the sleeve 30. (The term “yarn” as used herein is a generic term for a continuous strand or strands of filaments, fibers or other material in a form suitable for knitting, weaving, braiding or otherwise interlacing. Yarns include a number of fibers twisted together, a number of filaments laid together without twist, a number of filaments laid together with more or less twist, a monofilament, as well as strips or ribbons made by a lengthwise division of a sheet material.) Plied yarns comprise adjacent yarns which are woven into the fabric as one and can be formed in the warp direction by coordinating the movements of adjacent heddles to be the same during weaving. Plied yarns are formed in the fill direction by sending the shuttle through the same shed more than once in what is known as a “dead pick” operation, which lays multiple yarns adjacent to one another where normally there would be only one yarn. The dead pick operation is sequenced to occur when the fill yarns at or near the junction region are being interwoven.

Plied yarns increase the strength of the fabric in the area around where they are positioned because they provide a localized increase in the cross-sectional area over which to distribute the tensile stresses experienced by the fabric when it is subjected to external forces, such as the repeated pulsations of the hemodynamic pressure loads seen by a graft in the aorta.

Strengthening elements 32 and 34 can also comprise yarns of the same material as used to form the sleeve but having an increased denier. For example, a bifurcated sleeve woven of 40 denier yarns may have strengthening elements 32 and 34 comprising 80 denier yarns interwoven in the warp and fill directions. Preferably, the larger denier yarns comprise relatively few of the total number of yarns forming the sleeve so as not to significantly increase the bulk of the sleeve. The larger denier warp and fill yarns are positioned to cross one another in the junction region 28 as depicted in FIG. 2. To this end, the larger denier warp yarns 32 which run in the warp direction must be positioned appropriately when the loom is set up so that they pass through the junction region during weaving. The larger denier fill yarns 34 are carried on a separate shuttle which is passed through the shed at the appropriate time in the weaving process to position the larger denier fill yarns in the junction region.

Strengthening elements 32 and 34 may also comprise yarns of different material having greater tensile strength than the material used to form the yarns comprising the bifurcated sleeve 30. For example, when used in a stent graft to repair aortic aneurysms, bifurcated sleeve 30 may be made of polyester due to that material's compatibility with human tissue and long history of success in surgical implants. The polyester sleeve may be reinforced at the junction region 28 by strengthening elements 32 and 34 formed of a higher tensile strength material such as nylon or metal wire comprising stainless steel, nitinol or another metal compatible with human tissue. Such higher strength elements can be readily interwoven and positioned within the sleeve to reinforce the junction region, providing filamentary members of increased strength precisely at the weak point of the bifurcated sleeve. For applications where compatibility with human tissue is not a requirement, other high strength materials, such as Kevlar®, may also be considered. Incorporation of the higher strength elements into the sleeve is accomplished similarly as described above for the larger denier reinforcing elements.

A practical example of a bifurcated sleeve for use with a stent graft may be woven of 40 denier polyester yarns with the strengthening elements preferably comprising plied yarns of the same material and denier. This embodiment is preferred because it requires no special set-up procedures, no additional types of yarns or filaments and will not result in fill thread ends which must be trimmed when the bifurcated sleeve is removed from the loom, as would be necessary when different material is laid into the fill.

In the present example, four warp strengthening elements 32 are incorporated into the design on each side of the bifurcated sleeve, two elements being on each branch tube on each side. The warp strengthening elements 32 are plied by moving adjacent heddles, through which the strengthening yarns run, together as each shed is formed, causing two adjacent warp yarns to be woven as one 2-ply warp yarn relative to the fill. In the junction region 28, at about 15 sheds before the bifurcation point 36 is reached, a double fill insertion is made via a dead pick which forms a 2-ply 40 denier fill yarn comprising the first fill strengthening element 34a. The normal weave proceeds through about ten more sheds and a second dead pick is laid in forming another 2-ply 40 denier yarn, 34b, in the junction region closer to the bifurcation point. The 2-ply fill yarns 34 cross over the 2-ply warp yarns 32 within the junction region 28 to reinforce this otherwise weak area of the bifurcated sleeve. Should a tear in the fabric develop in the junction region, for example, at the bifurcation point 36, its propagation will be stopped in either the warp or fill directions when the tear reaches one of the strengthened elements which will not fail at the same stress level as the surrounding yarns comprising the sleeve.

### Knitted Bifurcated Sleeve Having Junction Region Strengthening Elements

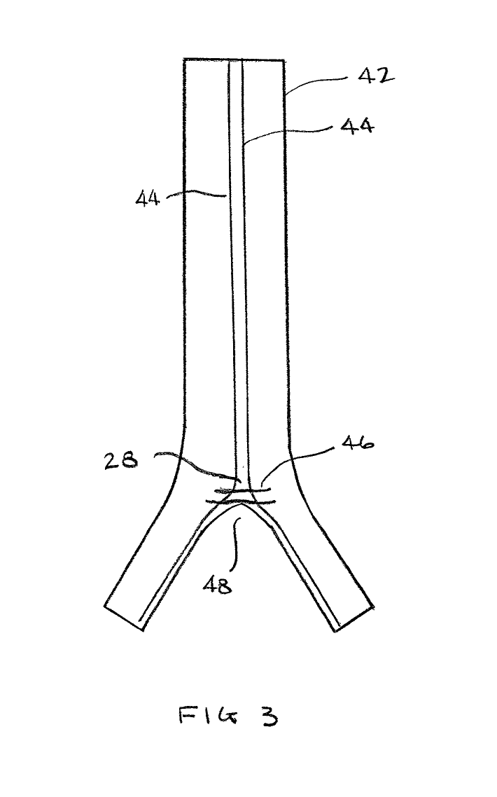

FIG. 3 shows a warp knitted bifurcated sleeve 42 having warp strengthening elements 44 arranged parallel to the warp direction of the sleeve and fill strengthening elements 46 intersecting the warp strengthening elements within the junction region 28 at or near the bifurcation point 48.

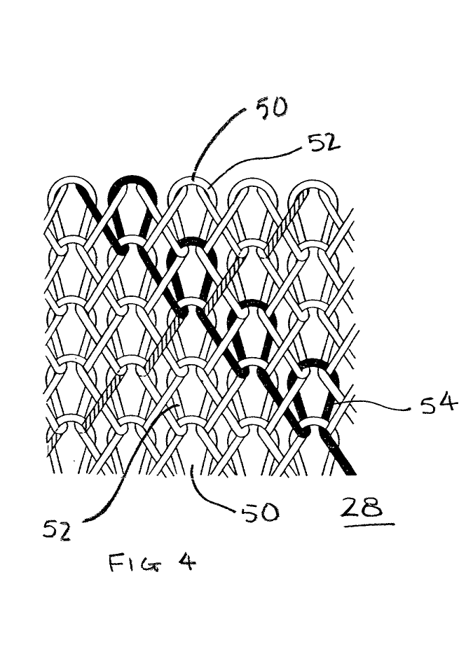

For the knitted sleeve 42, the warp strengthening elements 44 comprise a wale or column of loops 50 (shown in detail in FIG. 4 which depicts a portion of the junction region 28 of FIG. 3 on an enlarged scale) made of yarns or filaments 52 which are, in some way, stronger than the yarns or filaments comprising the other wales of the sleeve. Analogously to the woven sleeve described above, the loops 50 comprising a strengthening element 44 may comprise yarns or filaments 52 of the same material as used to form the rest of the sleeve but having an increased denier to yield greater tensile strength. In another embodiment, two or more yarns of the same material as used to make the sleeve may be plied together and used to form the loops 50 comprising the reinforcing elements. The yarns or filaments 52 forming the loops comprising the strengthening element may also be formed from a different, stronger material than the rest of the sleeve. Like the woven sleeve, the warp reinforcing elements 44 are located within the sleeve by positioning the strengthening yarns on the knitting machine so that the needles which will be knitting the junction region 28 engage those yarns as the courses are knitted.

Fill strengthening elements 46 are formed by controlling the action of the needles forming the strengthening elements in the fill direction as they knit the junction region 28. For most of the length of the sleeve 42, the needles are not moved significantly in the fill direction except as required to intermesh the loops. However, within the junction region 28 the needles engaging the yarns or filaments 52 are moved significantly to knit these strengthened filamentary members in the fill direction thus forming strengthening elements 46 within the junction region.

The action of the needles may also be controlled when knitting in the region of the junction region to effect a different type of knit. For example, as shown in FIG. 4, a locking knit 54 may be used to create the strengthening elements 46 and 44 within the junction region, or the density of the knit may be changed by adding more courses per inch. Note that the locking stitch is oriented angularly relatively to courses 50.

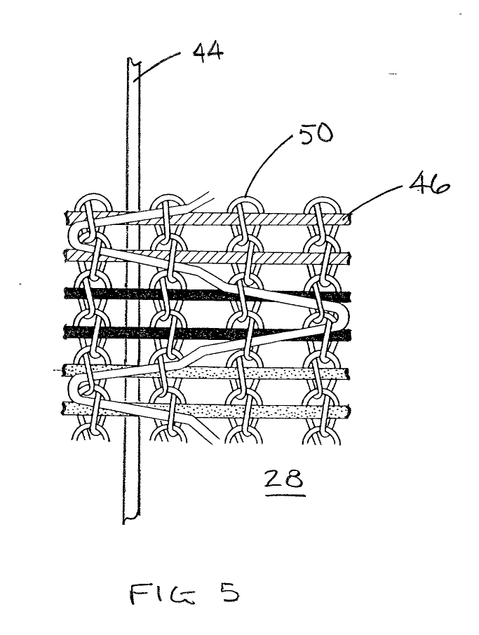

As an alternative, the strengthening elements 44 and 46 may be laid into the knit structure as illustrated in FIG. 5, which also depicts a portion of the junction region 28 from FIG. 3 on an enlarged scale. Warp strengthening elements 44 proceed lengthwise along the sleeve and intersect the fill strengthening elements 46 in the junction region 28. As in the woven embodiment, the strengthening elements may be plied yarns, yarns made from material having relatively high tensile strength or yarns having relatively larger denier.

Concentrating the strengthening elements at the known weak point in the bifurcated sleeve in the manner according to the invention provides the following advantages: (1) the bulk of the sleeve is not significantly affected, allowing a woven sleeve, reinforced in this manner, to still be implanted in the vascular system through a catheter; (2) relatively few strengthening elements are needed, making economical use of the more expensive, higher strength yarns and filaments; (3) fewer special steps are required in the knitting or weaving process, for example, the fewer different yarns are used the fewer times they need to be switched in and out of the weaving process.

The bifurcated fabric sleeve according to the invention promises to yield a strengthened, more reliable, longer lasting and relatively economical graft for the repair of life threatening aneurysms.

What is claimed is:

1. In a stent graft, a bifurcated sleeve formed of interlaced filamentary members, said sleeve comprising:
an elongated flexible first tubular member;
at least one elongated flexible second tubular member joined to said first tubular member;
a junction region formed of said interlaced filamentary members, said junction region being positioned between and joining said first and second tubular members; and
an elongated strengthening element interlaced with said filamentary members forming said junction region, said strengthening element having a relatively greater tensile strength than said filamentary members for reinforcing said junction region.

2. A bifurcated sleeve according to claim 1, wherein said strengthening element is positioned substantially lengthwise along one of said first and second tubular members.

3. A bifurcated sleeve according to claim 2, further comprising another elongated strengthening element having a relatively greater tensile strength than said filamentary members for reinforcing said junction region, said other strengthening element being interlaced with said filamentary members forming said junction region and being oriented angularly with respect to said strengthening element, said strengthening elements intersecting one another within said junction region.

4. A bifurcated sleeve according to claim 3, wherein said filamentary members and said strengthening elements are interlaced by weaving.

5. A bifurcated sleeve according to claim 4, wherein one of said strengthening elements comprises plied filamentary members interwoven with said filamentary members comprising said sleeve.

6. A bifurcated sleeve according to claim 5, wherein said plied filamentary members have substantially the same denier and comprise substantially the same material as the filamentary members comprising said sleeve.

7. A bifurcated sleeve according to claim 4, wherein one of said strengthening elements comprises a reinforcing filamentary member formed from a material having a relatively greater tensile strength than the material forming said filamentary members comprising said sleeve.

8. A bifurcated sleeve according to claim 4, wherein one of said strengthening elements comprises a reinforcing filamentary member having a relatively greater denier than said filamentary members comprising said sleeve.

9. A bifurcated sleeve according to claim 3, wherein said filamentary members are interlaced by knitting to form a plurality of wales and courses comprising said sleeve, said strengthening element comprising a wale of said sleeve and said other strengthening element comprising a course of said sleeve.

10. A bifurcated sleeve according to claim 9, wherein one of said strengthening elements comprises plied filamentary members.

11. A bifurcated sleeve according to claim 10, wherein said plied filamentary members have substantially the same denier and comprise substantially the same material as the filamentary members comprising said sleeve.

12. A bifurcated sleeve according to claim 9, wherein one of said strengthening elements comprises a reinforcing filamentary member formed from a material having a relatively greater tensile strength than the material forming said filamentary members comprising said sleeve.

13. A bifurcated sleeve according to claim 9, wherein one of said strengthening elements comprises a reinforcing filamentary member having a relatively greater denier than said filamentary members comprising said sleeve.

14. A bifurcated sleeve according to claim 9, wherein one of said wale and said course is knitted in a locking stitch.

15. A bifurcated sleeve according to claim 9, further comprising a plurality of said strengthening elements comprising a plurality of said wales and a plurality of said courses, said wales and courses comprising said strengthening elements forming said junction region.

16. A bifurcated sleeve according to claim 15, wherein said plurality of wales and courses comprising said strengthening elements are knitted with a relatively greater density per unit area than said wales and courses otherwise comprising said sleeve.

17. A bifurcated sleeve according to claim 2, wherein said filamentary members are interlaced by knitting to form a plurality of wales and courses comprising said sleeve, said strengthening element comprising a first reinforcing filamentary member laid in with said wales and courses.

18. A bifurcated sleeve according to claim 17, wherein said other strengthening element comprises a second reinforcing filamentary member laid in with said wales and courses.

19. A bifurcated sleeve according to claim 18, wherein one of said strengthening elements comprises plied filamentary members.

20. A bifurcated sleeve according to claim 19, wherein said plied filamentary members have substantially the same denier and comprise substantially the same material as the filamentary members comprising said sleeve.

21. A bifurcated sleeve according to claim 18, wherein one of said strengthening elements comprises a reinforcing filamentary member formed of a material having a relatively greater tensile strength than the material forming said filamentary members comprising said sleeve.

22. A bifurcated sleeve according to claim 18, wherein one of said strengthening elements comprises a reinforcing filamentary member having a relatively greater denier than said filamentary members comprising said sleeve.

23. In a stent graft, a bifurcated sleeve formed of interlaced filamentary members, said sleeve comprising:
an elongated flexible first tubular member;
two second flexible tubular members extending from one end of said first tubular member;
a junction region positioned between and joining said second tubular members together;
an elongated first strengthening element interlaced with said filamentary members forming said junction region, said first strengthening element being arranged substantially lengthwise along said first tubular member and traversing said junction region and one of said second tubular members lengthwise therealong, said first strengthening element having a relatively greater tensile strength than said filamentary members for reinforcing said junction region; and
an elongated second strengthening element interlaced with said filamentary members forming said sleeve, said second strengthening element traversing said junction region and being oriented angularly with respect to said first strengthening element, said second strengthening element having a relatively greater tensile strength than said filamentary members for reinforcing said junction region.

24. A bifurcated sleeve according to claim 23, further comprising a plurality of said first and second strengthening elements.

25. A bifurcated sleeve according to claim 24, wherein one of said strengthening elements comprises plied filamentary members.

26. A bifurcated sleeve according to claim 24, wherein one of said strengthening elements comprises a filamentary member having a relatively larger denier than said filamentary members forming said sleeve.

27. A bifurcated sleeve according to claim 24, wherein one of said strengthening elements comprises a filamentary member formed of a material having a relatively greater tensile strength than the material forming said filamentary members forming said sleeve.

28. A bifurcated sleeve according to claim 24, wherein said filamentary members are interlaced by weaving.
